(19) United States
(12) Patent Application Publication
(10) Pub. No.: US 2007/0255305 A1
(43) Pub. Date: Nov. 1, 2007

(54) Percutaneous dilation apparatus

(76) Inventors: Donald J. McMichael, Roswell, GA (US); Nathan V. Shirley, Herriman, UT (US); Kristy Peterson, Santa Rosa, CA (US)

Correspondence Address: KIMBERLY-CLARK WORLDWIDE, INC.;Catherine E. Wolf, 401 NORTH LAKE STREET, NEENAH, WI, 54956, US

(21) Appl. No.: 11/413,577

(22) Filed: Apr. 28, 2006

(51) Int. Cl. A61M 29/00

(52) U.S. Cl. 606/191

(57) ABSTRACT

A percutaneous dilation apparatus is shown. The apparatus is useful for forming and enlarging percutaneous penetrations to a variety of target locations within a patient's body for multiple purposes. The apparatus includes an elongate dilation tube, a plurality of elongate expansion members disposed in a nested concentric arrangement around the elongate expansion tube, and an outer sheath for capturing the plurality of elongate expansion members and maintaining the apparatus in an assembled configuration. Each expansion member is independently movable from a first to a second position along the central axis of the tube as well as each concentrically smaller member. A trocar may be provided for slidably engaging the lumen through the elongate dilatation tube.

### BACKGROUND OF THE INVENTION

The present invention relates generally to a medical device for facilitating the percutaneous access of a body lumen and, more particularly, it relates to the construction and use of a dilator tool or dilation apparatus which enables the sequential radial dilation of a tissue opening to create larger diameter working channels into the body lumen.

Modern medicine frequently requires percutaneous access to hollow body organs, tissue, cavities, and the like. In the case of “least or minimally invasive” surgical procedures, such access is usually provided by inserting a suitable cannula, instrument, tube, or the like, through a small access hole. The initial access is usually created by piercing the skin and any intermediate body structures with a needle or trocar. The initial puncture, however, is usually very small so that the needle or trocar can achieve the desired penetration without excessive damage to tissue. It is therefore necessary for the initial access hole to be subsequently enlarged to provide a working channel having a sufficient diameter to permit performance of the desired medical procedure.

One common technique for achieving such enlargement relies on successively introducing one or more dilating rods having increasingly larger diameters through the puncture hole and into the body organ, tissue, or cavity. When a flexible guide wire has been introduced through the initial needle or cannula puncture, this protocol is referred to as the Seldinger technique.

While this technique is reasonably effective for placement of relatively small devices, e.g., catheters to about 6 French [1 French (F) is equal to 0.079 inch diameter], larger dilations require increasing numbers of dilator exchanges and can be extremely time consuming. Moreover, the body structures that are being penetrated frequently comprise relatively flaccid membranes or walls so that penetration with larger dilators may cause fascial detachment, i.e., the invagination and separation of the membrane or wall from surrounding tissue structures. Such problems may be exacerbated when the organ, tissue, or cavity being penetrated is diseased so that the membranes or walls are thickened or toughened and resistant to penetration by the dilator which axially engages the tissue.

One approach for preventing fascial detachment of the internal body organ or structure during the dilation process involves the use of separate anchoring instruments which are placed around the site of penetration and dilation. The technique, developed by Dr. Cope, relies on the placement of multiple separate anchors or toggles peripherally about the site of the primary puncture in order to more strongly attach the body organ to its surrounding fascia. The anchors are attached to lengths of suture which extend through the tracks defined by the separate punctures. The sutures are tensioned in order to hold the wall of the hollow organ against the fascia and subsequently secured outside the body. While this approach is generally successful, it requires a separate puncture for each anchor and the subsequent suturing of each anchor in place. The technique is therefore relatively time consuming, costly, and potentially subjects the patient to greater discomfort.

An additional problem with the use of successively larger dilators, either with or without use of an anchoring technique, is the leakage of body fluids and substances through the penetration which is being enlarged. While such leakage will be inhibited while each successive dilator is in place, removal of the dilator will allow the fluids from the organ, tissue or cavity being penetrated to contaminate other body structures on the puncture track. For example, percutaneous access to the gallbladder is normally achieved transhepatically since the gallbladder is partially attached to the liver. Transperitoneal access proceeds through an unattached wall of the gallbladder and increases the likelihood of bile leakage into the peritoneal. While transperitoneal access might otherwise be preferred for a number of reasons, e.g., it avoids potential damage to the liver, it is contraindicated by the difficulty in penetrating the unattached wall of the gallbladder and the greater risk of bile leakage associated with conventional dilation techniques.

For these reasons, it would be desirable to provide improved methods and apparatus for forming and enlarging percutaneous penetrations into hollow body organs, tissues, and cavities. The apparatus and methods should be suitable for enlarging percutaneous access penetrations to virtually any diameter, including very large diameters on the order of 20 F, 24 F, and larger while reducing the risk of invagination and fascial detachment of the organ, tissue, or cavity which is being penetrated. The methods should minimize any additional time and complexity required for performing an associated interventional procedure, and in particular, should avoid the need to make secondary penetrations in order to secure the body organ, tissue, or cavity to surrounding fascia. The methods should further avoid complexity and will preferably reduce the number of dilation mechanisms required to achieve a desired enlargement. The method should also lessen the patient discomfort associated with the procedure and should be compatible with virtually any type of interventional procedure which requires the formation of a percutaneous penetration for access to the body organ, tissue, or cavity.

### SUMMARY OF THE INVENTION

In response to the foregoing problems and difficulties encountered by those of skill in the art, the present invention is directed toward a percutaneous dilation apparatus. In one aspect of the invention, the percutaneous dilation apparatus may include an elongate dilation tube having a proximal end, a distal end, and an axial lumen defining a path through a central axis of the tube. A plurality of elongate expansion members are disposed in a nested concentric arrangement with respect to the elongate expansion tube which is centrally disposed within a lumen of the innermost elongate expansion member. Each expansion member is independently movable from a first to a second position along the central axis of the tube as well as each concentrically smaller member. An outer sheath is attached to the elongate dilation tube proximal to the distal end. The sheath is for capturing the plurality of elongate expansion members and maintaining the apparatus in an assembled configuration. A trocar may be provided for slidably engaging the lumen through the elongate dilatation tube.

In other embodiments, at least a portion of the dilation tube and/or expansion members may be made flexible. A lubricious coating may be applied to the dilation tube, expansion members, or both. In one alternative, the dilation tube, expansion members, or both may be made of or contain a lubricious polymer.

In certain embodiments, each expansion member has a length which is generally less than that of the dilation tube. Moreover, each expansion member may comprise a tapered distal end. In such an embodiment, each distal end of each expansion member is tapered, and the serial arrangement provides a continuously larger cross-sectional area of the apparatus until a portion of the outermost expansion member is reached.

Other embodiments may possess a plurality of tabs, each tab being associated with each expansion member, may be provided. Each tab would be used for grasping and manipulating its expansion member from the first to the second position. Manipulation of each of the expansion members to its respective second position enables a distal end of each to be serially disposed and accessible along a portion of the dilation tube.

Other objects, advantages and applications of the present invention will be made clear by the following detailed description of a preferred embodiment of the invention and the accompanying drawings wherein reference numerals refer to like or equivalent structures.

### BRIEF DESCRIPTION OF THE DRAWINGS

FIG. 1 is a perspective view of one embodiment of the present invention;

FIG. 2 is a cutaway of the FIG. 1 view taken through the longitudinal center line;

FIG. 3 is a perspective view of the FIG. 1 embodiment in a partially extended arrangement;

FIG. 4 depicts an exemplary embodiment of an expansion member used in the FIG. 1 embodiment separated from the apparatus for clarity; and

FIG. 5 is a perspective view of the proximal end of the FIG. 1 device depicting the sheath portion and tabs.

### DETAILED DESCRIPTION

The present invention is useful for forming and enlarging percutaneous penetrations to a variety of target locations within a patient's body for a multiplicity of purposes. The initial penetration will be very small, usually being below about 7 F, more usually being below about 3 F, and frequently being below about 20 GA (gauge; 0.035 in). The penetration will subsequently be enlarged to a desired final size, usually having a final diameter in the range from about 10 French (F) to about 30 F, typically being from about 12 F to 28 F, and usually being from about 14 F to 24 F, with the present invention being particularly useful for the formation of larger diameter penetrations.

The purpose of the penetration may be for drainage, intraorgan drug administration, perfusion, aspiration, or the like, but will usually be for the introduction of a relatively large surgical instrument or working catheter, such as those intended for least invasive surgical procedures. Such procedures include laparoscopy, balloon dilation of ducts, placement of stents, urological and biliary stone removal, ostomy procedures such as colonoscopy, tracheostomy, and the like. Another common purpose for the penetration may be for feeding directly to the gastrointestinal tract such as via a jejunostomy or gastrostomy. Target locations for the percutaneous penetrations will usually be the interior of a hollow body organ or body cavity, such as the gallbladder, stomach, urinary bladder, uterus, kidney, portions of the lung and trachea, rectum, the peritoneum, and the like. The target locations may also be situated within solid tissue as well as solid organs, such as a solid tumor or abscess. Depending on the location which is being accessed, the length and flexibility of the apparatus of the present invention may vary significantly.

A percutaneous dilation apparatus or tool according to the present invention includes an elongate dilation tube having an axial lumen which defines a path through a central axis of the elongate dilation tube. The elongate dilation tube will have proximal and distal ends, and may have a generally flexible or rigid structure, depending on the particular application. Rigid or semi-rigid dilation tubes will generally be employed when the target organ may be approached along a substantially straight path, while more flexible dilation tubes will be employed when the access route is more tortuous.

The length of the elongate dilation tube will vary, with shorter dilation tubes typically having a length in the range from about 7 cm to 12 cm and being suitable for accessing target locations which are near the surface of the skin, such as the stomach or trachea. Longer dilation tubes will have a length in the range from about 15 cm to 25 cm and will be suitable for accessing more remote target locations, such as the kidney. Even longer flexible dilation tubes having lengths in the range from about 30 cm to 50 cm, or longer, may be employed for accessing the most remote ducts and body locations.

A penetration device having a sharpened tip will optionally be provided in conjunction with the elongate dilation tube for puncturing the skin and underlying tissue, organs, and the like, as the tube is percutaneously advanced toward its target location. Conveniently, the axial lumen through the elongate tube provides a passage for the introduction of the penetration device in the form of a needle, stylet, or trocar. The penetration device is placed into the axial lumen of the dilation tube so that the sharpened tip is exposed at the distal end of the combined assembly. The assembly may then be percutaneously advanced to the target location and the penetration device removed prior to radial expansion of the tool, as described hereinafter.

A sharpened tip or other means for puncturing the skin will be necessary when no previous needle puncture would have been made. The present invention, however, is useful in cases where conventional techniques and apparatus are used to form an initial, relatively small diameter, puncture. Typically, the puncture will be made using a very small needle, and it will be possible in some cases to introduce the dilation tube of the present invention (without a sharpened tip) directly into the initial puncture track. More commonly, the initial puncture track will be subsequently enlarged to an intermediate diameter using conventional techniques and apparatus, such as the Seldinger technique combined with very small axial dilation. The dilated intermediate diameter will typically be in the range from about 3 F to 8 F, more typically being in the range from about 5 F to 7 F. The dilation tube of the present invention may then be introduced into the partially dilated penetration, typically over a flexible guide wire or other member which has been left in place to maintain the track. The penetration may then be enlarged by the subsequent axial introduction of the expansion member or members in order to achieve the final desired diameter for the access lumen.

Each elongate expansion member will have a length which is generally less than that of the dilation tube, and will have an outer diameter which is larger than the diameter of the dilation tube. In many cases, the procedure to be performed will employ two or more dilation members having successively larger diameters to provide for an incremental expansion, however, in some procedures the diameter of a single dilation member will be sufficient to radially expand the stoma to its final desired diameter. Typically, the outer diameter of the largest expansion member will be at least two fold larger than the diameter of the dilation tube lumen, usually being at least three fold larger, and frequently being five fold or more larger.

The elongate expansion member will usually have a tapered distal end to facilitate advancement of the member through the patient's anatomy. Additionally, the exterior surface of the expansion member may be wholly or partly coated with a lubricant to further facilitate penetration or be manufactured from a lubricious polymer such as polyethylene, although this may not be necessary when the inner surface of the axial lumen of the tube is itself lubricous.

Most simply, the elongate expansion member may be a cannula having a single axial lumen which defines a path through a central axis of the elongate expansion member, the elongate dilation tube slidably engaging the axial lumen of the elongate expansion member. Manipulation of the elongate expansion member in a distal direction serves to slide the expansion member over the dilation tube. This operates to increase the outer diameter of the tool and axially expand a stoma within which the tool is positioned. Subsequent expansion members are concentrically assembled about the dilation tube and first expansion member in a similar manner.

An outer sheath is provided as the outermost concentrically disposed cannula. The sheath provides a user manipulable portion of the tool and serves to maintain the concentrically stacked arrangement of expansion members around the dilation tube. After the final required expansion member is slid into place over the dilation tube and the stoma is axially expanded as desired, the tool may then be removed and the ultimate procedure performed.

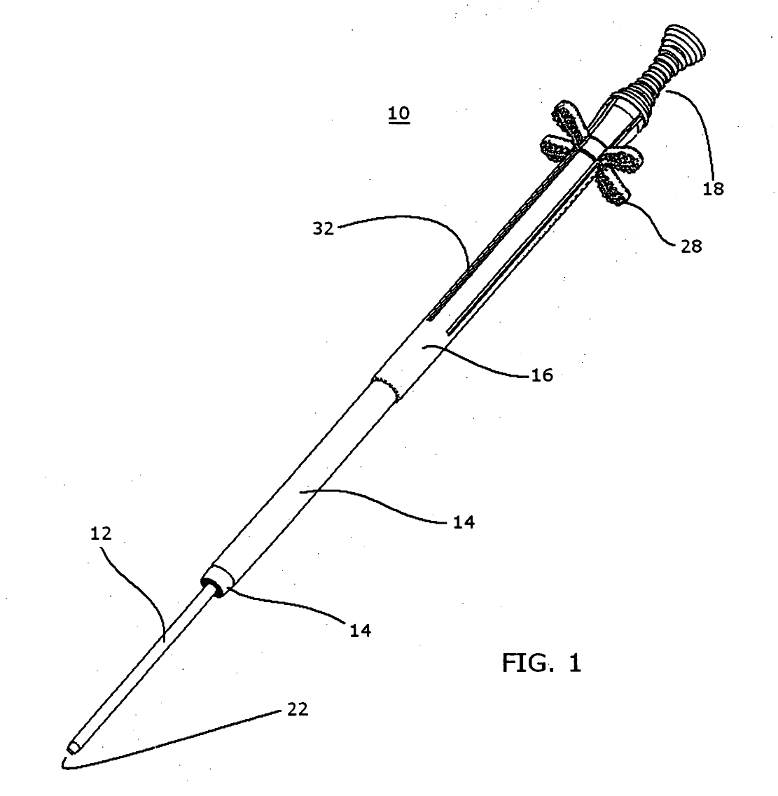

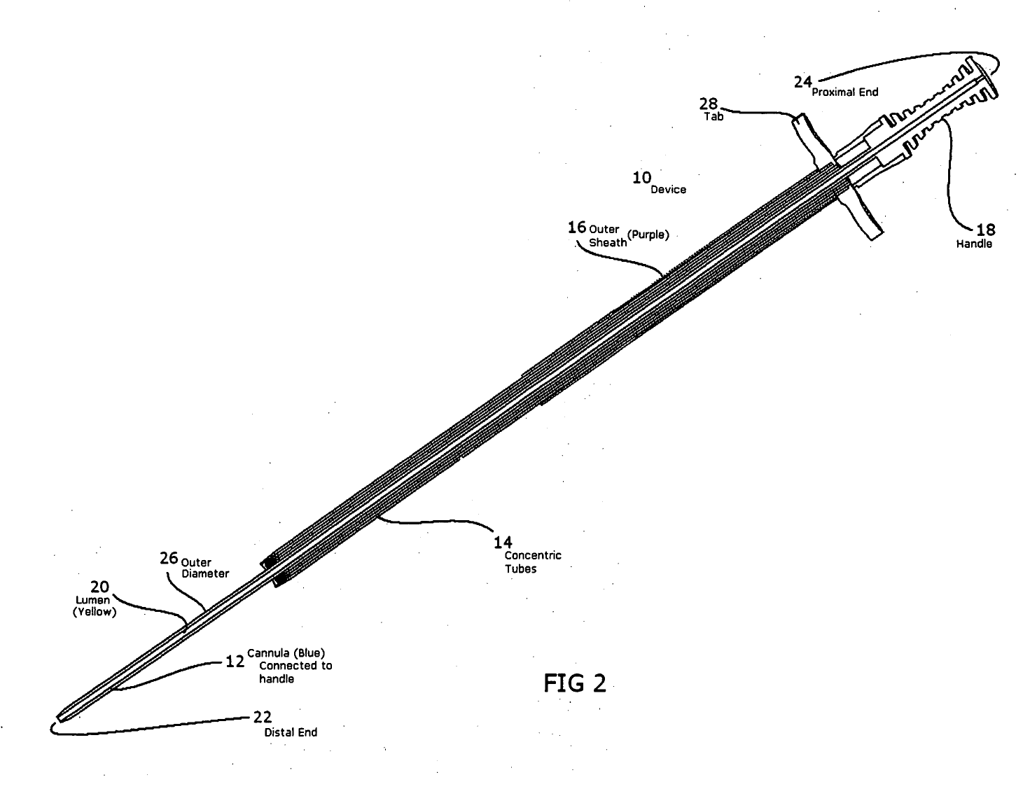

Referring now to FIGS. 1 and 2, a percutaneous dilation apparatus or tool 10 constructed in accordance with the principles of the present invention will be described in a first exemplary embodiment as a gastrointestinal tube placement tool. The apparatus 10 includes an elongate dilation tube 12, at least one elongate expansion member 14, and an outer sheath 16. A handle 18 is provided to enable a physician to manipulate the tool 10. The dilation tube 12 comprises an axial lumen 20 through which a penetration device (not shown) is introduced into the patient. The dilation tube 12 also has a distal end 22, a proximal end 24, and an outer diameter 26. The outer diameter 26 of the dilation tube 12, the lumen 20, or both may be made lubricious or manufactured of lubricious polymers to decrease friction between various of the components. The handle 18 may be formed into the proximal end 24 of the dilation tube 12.

The elongate expansion member 14 or plurality of members 14 is disposed in a stacked or nested and concentrically oriented arrangement about the elongate dilation tube 12. As such each successively larger diameter elongate expansion member 14 envelops the next smaller member 14. Each member 14 is capable of sliding along its central axis independent of its neighbor. To manipulate each member 14 accordingly, a tab 28 is provided proximal to the handle 18. The tab is formed as a proximal axial extension of the expansion member. The tab 28 enables the clinician to move the elongate expansion member in a forward or backward direction along the axis of the dilation tube 12.

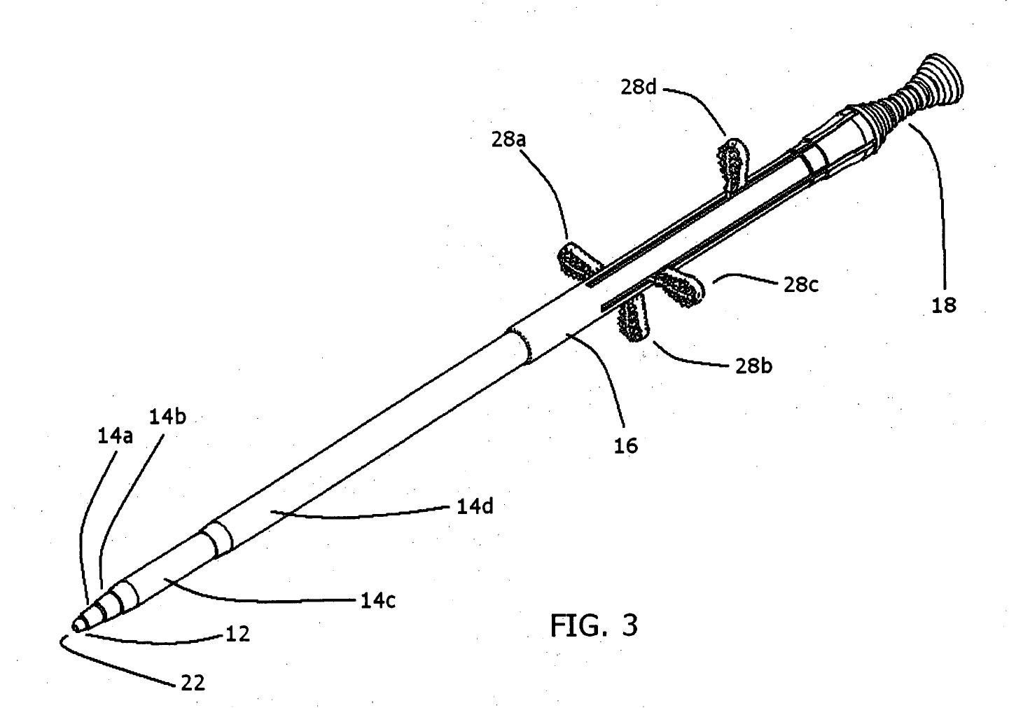

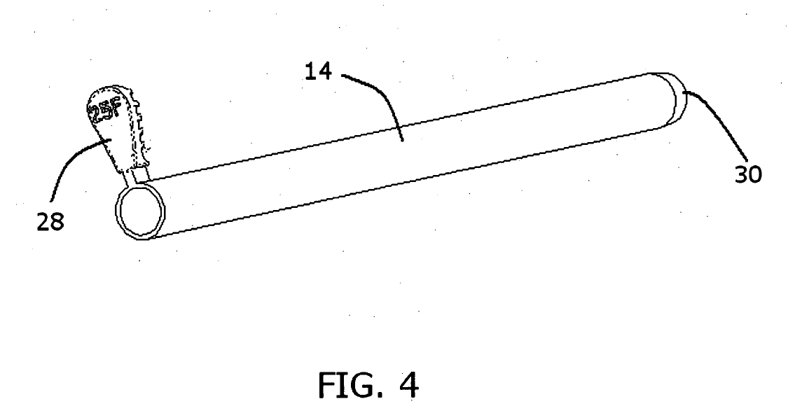

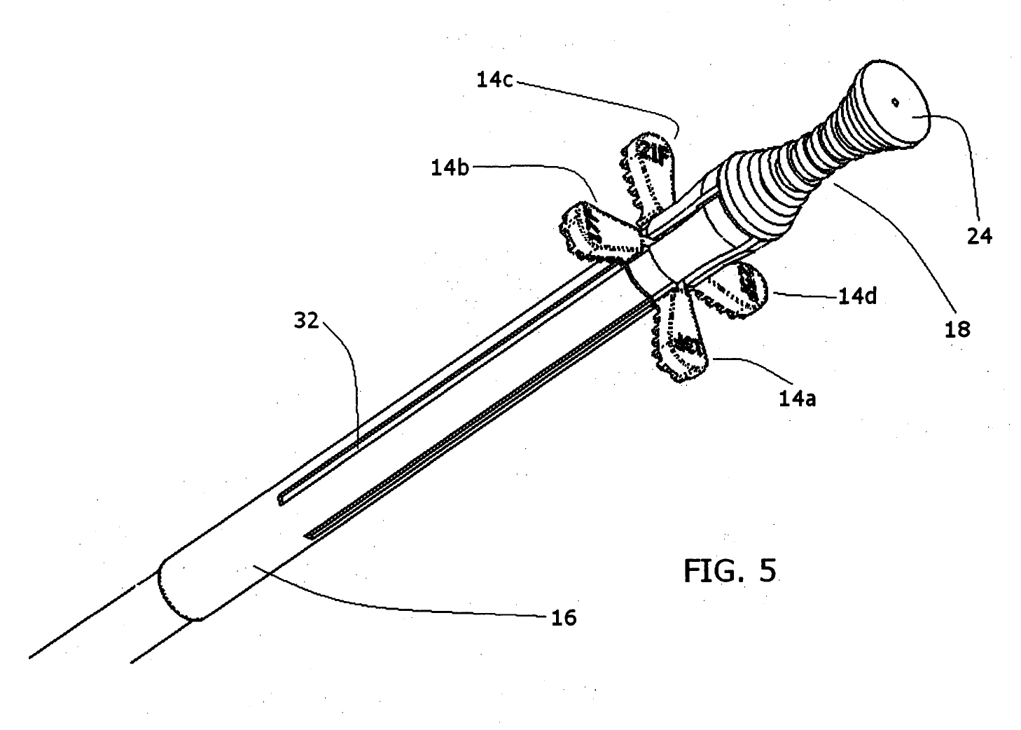

Looking at the embodiment in FIG. 3, it may be seen that the tool 10 possesses four elongate expansion members, 14a, 14b, 14c, and 14d each controlled by its own tab 28a, 28b, 28c, and 28d, respectively. In the FIG., it may be seen that expansion members 14a, 14b, and 14c are fully deployed whereas expansion member 14d is partially deployed. It should be noted that in this embodiment, each expansion member has a tapered distal end 30 to facilitate introduction and gradual expansion of the stoma. Additionally, either or both of the interior and exterior surfaces of each expansion member may be wholly or partly coated with a lubricant to further facilitate penetration. FIG. 4 depicts an exemplary embodiment of one such expansion member 14 separated from the apparatus for clarity. In one embodiment as depicted in FIG. 5, expansion member 14a may be about 13 F, expansion member 14b may be about 17 F, expansion member 14c may be about 21 F, and expansion member 14d may be about 25 F.

An outer sheath 16 is provided as the outermost concentrically disposed cannula and serves in part to maintain the concentrically stacked arrangement of expansion members 14 around the dilation tube 12. The sheath 16 contains a plurality of slots 32, each slot 32 associated with a tab 28. The slot 32 enables the elongate expansion member 28 to slide from a first to a second position and as such accommodates the tab associated with any given expansion member. The slot length may be individually designed to limit the travel of each expansion member 28 so as to place the tapered distal end 30 of each member 28 in a desired location such as that shown in FIG. 3 where the tapers cooperate to provide the tool 10 with an overall tapered distal end 22. The sheath 16 may be attached to a portion of the handle 18 on the dilation tube 12 such that the expansion members 14 are captured accordingly. Appropriately a quantity of the elongate expansion members 14 may also possess slots in alignment with those slots 32 in the sheath 16 in order to fully accommodate movement of other expansion members 14.

As used herein and in the claims, the term “comprising” is inclusive or open-ended and does not exclude additional unrecited elements, compositional components, or method steps.

While various patents may have been incorporated herein by reference, to the extent there is any inconsistency between incorporated material and that of the written specification, the written specification shall control. In addition, while the invention has been described in detail with respect to specific embodiments thereof, it will be apparent to those skilled in the art that various alterations, modifications and other changes may be made to the invention without departing from the spirit and scope of the present invention. It is therefore intended that the claims cover all such modifications, alterations and other changes encompassed by the appended claims.

We claim:

1. A percutaneous dilation apparatus comprising:
an elongate dilation tube having a proximal end, a distal end, and an axial lumen defining a path through a central axis of the tube;
a plurality of elongate expansion members disposed in a nested concentric arrangement with respect to the elongate expansion tube which is centrally disposed within a lumen of the innermost elongate expansion member, each expansion member being independently movable from a first to a second position along the central axis of the tube and each concentrically smaller member; and
an outer sheath attached to the elongate dilation tube proximal to the distal end, the sheath for capturing the plurality of elongate expansion members and maintaining the apparatus in an assembled configuration.

2. The apparatus of claim 1 comprising a trocar slidably engageable with the lumen through the elongate dilatation tube.

3. The apparatus of claim 1 wherein the at least a portion of the dilation tube and expansion members are flexible.

4. The apparatus of claim 1 wherein each expansion member has a length which is generally less than that of the dilation tube.

5. The apparatus of claim 1 wherein each expansion member comprises a tapered distal end.

6. The apparatus of claim 1 comprising a lubricious coating upon at least the dilation tube or expansion members.

7. The apparatus of claim 1 wherein at least one of the dilation tube or expansion members contain a lubricious polymer.

8. The apparatus of claim 1 comprising a plurality of tabs, a tab associated with each expansion member, each tab for grasping and manipulating its expansion member from the first to the second position.

9. The apparatus of claim 1 wherein manipulation of each of the expansion members to its respective second position enables a distal end of each to be serially disposed and accessible along a portion of the dilation tube.

10. The apparatus of claim 8 wherein each distal end of each expansion member is tapered, and the serial arrangement provides a continuously larger cross-sectional area of the apparatus until a portion of the outermost expansion member is reached.

11. A percutaneous dilation apparatus comprising:
an elongate dilation tube having a proximal end, a distal end, and an axial lumen defining a path through a central axis of the tube;
at least one elongate expansion member having an axial lumen defining a path through a central axis of the expansion member, the axial lumen of the expansion member for slidingly receiving the elongate dilation tube therein; and
an outer sheath for capturing the elongate dilation tube and the at least one elongate expansion member and enabling them to move with respect to one another.

12. The apparatus of claim 11 comprising a tab associated with the expansion member, the tab for grasping and manipulating the expansion member from a first to a second position.

13. The apparatus of claim 12 wherein the outer sheath comprises a slot for slidable engagement of the tab therein.
